(19) United States
(12) Patent Application Publication
(10) Pub. No.: US 2018/0180595 A1
(43) Pub. Date: Jun. 28, 2018

(54) CELL LINES MUTANT FOR PATCHED 1 AND PATCHED 2 AND METHODS OF USE THEREOF

(71) Applicant: The Regents of the University of California, Oakland, CA (US)

(72) Inventor: Henk Roelink, Berkeley, CA (US)

(21) Appl. No.: 15/739,933

(22) PCT Filed: Jul. 21, 2016
(86) PCT No.: PCT/US16/43415

§ 371 Date: Dec. 26, 2017

Related U.S. Application Data
(60) Provisional application No. 62/196,760, filed on Jul. 24, 2015.

(51) Int. Cl. G01N 33/50; A61K 35/33; A61K 35/545; C12N 15/85; C12N 5/077

(52) U.S. Cl. CPC G01N 33/5041; A61K 35/33; A61K 35/545; C12N 2015/859; C12N 5/0656; C12N 2510/00; C12N 15/85

(57) ABSTRACT

The present disclosure provides a genetically modified cell, wherein the cell is genetically modified such that it does not produce functional Ptch1 and Ptch2 protein. The present disclosure provides screening methods to identify agents that modulate the Hedgehog (Hh) pathway. Agents of interest include those that inhibit the Hh pathway response of Ptch1^{-/-}; Ptch2^{-/-} cells, and those that further induce the Hh pathway of these cells.

### CROSS-REFERENCE

This application claims the benefit of U.S. Provisional Patent Application No. 62/196,760, filed Jul. 24, 2015, which application is incorporated herein by reference in its entirety.

### STATEMENT REGARDING FEDERALLY SPONSORED RESEARCH

This invention was made with government support under Grant No. R01GM097035 awarded by the National Institutes of Health. The government has certain rights in the invention.

### INTRODUCTION

Mammals produce three Hedgehog (Hh) proteins: Sonic Hedgehog (Shh), Indian Hedgehog (Ihh) and Desert Hedgehog (Dhh), of which Shh is the best studied. The Hh signaling pathway has evolutionarily conserved roles essential for normal embryonic development (Riddle et al., Cell. 1993, 75:1401-1416; Echelard et al., Cell. 1993, 75:1417-1430; Krauss et al., Cell. 1993, 75:1431-1444; Roelink et al., Cell. 1994, 76:761-775, 1994; Chang et al., Development. 1994, 120:3339-3353). For example, Shh activity regulates polarization in the limb bud (Lewis et al., Cell. 2001, 105:599-612) and plays roles in the notochord and floor plate of the neural tube (Roelink et al., Cell. 1994, 76:761-775; Roelink et al., Cell. 1995, 81:445-455). Hh signaling also plays crucial roles in the homeostasis of adult tissue (Zacharias et al., Dev Biol. 2011, 355:152-162), including the regulation of stem cell homeostasis (Adolphe et al., Development. 2004, 131:5009-5019).

Shh signaling is regulated by the interaction of three key components that include the Shh ligand, its receptor Patched 1 (Ptch1) (Marigo et al., Nature. 1996, 384:176-179; Stone et al., Nature. 1996, 384:129-134) and the pathway activator Smoothened (Smo) (Marigo et al., Nature. 1996, 384:176-179; Murone et al., Curr Biol. 1999, 9:76-84.). Under the prevailing model of Shh pathway activation, the binding of Shh to Ptch1 results in the release of Ptch1-mediated inhibition of Smo (Taipale et al., Nature. 2002, 418:892-897), leading to Smo activation and subsequent cell-autonomous activation of the Shh response (Huangfu and Anderson, Proc Natl Acad Sci USA. 2005, 102:11325-11330; Corbit et al., Nature. 2005, 437:1018-1021; Rohatgi et al., Science. 2007, 317:372-376). Smo mediates downstream signal transduction that includes dissociation of Gli transcription factors from kinesin-family protein Kif7 (Liem et al., Proc Natl Acad Sci USA. 2009, 106:13377-13382), and the key intracellular Hh pathway regulator Sufu (Varjosalo et al., Dev Cell. 2006, 10:177-186). Activated Gli transcription factors promote transcription of Hh target genes including Gli1 and Ptch1, both of which serve as readouts of Hh pathway activation (Chuang and McMahon, Nature. 1999, 617-621).

In addition to being essential for normal embryonic development and adult tissue homeostasis, aberrant Hh signaling has been found to be responsible for the initiation of a growing number of cancers (Scales and de Sauvage, Trends Pharmacol. Sci. 2009, 30:303-312). For example, classically, germline loss of function mutations of Ptch1 are found in Gorlin syndrome, characterized by the occurrence of basal cell carcinoma, medulloblastoma, and a predisposition to develop rhabdomyosarcoma and meningioma (Boutet et al., J Invest Dermatol. 2003, 121:478-481; Epstein, Nat. Rev. Cancer. 2008, 8:743-754). Recently, persistent Hh activity has been implicated in cancers of the gastrointestinal tract (e.g., lung, breast, liver, stomach, pancreas, etc.), as well as ovarian cancer (Di Magno et al., Biochim Biophys Acta. 2015, 1856:62-72).

There is a need in the art for methods of identifying agents that target the Hh pathway.

### SUMMARY

The present disclosure provides a genetically modified cell, wherein the cell is genetically modified such that it does not produce functional Ptch1 and Ptch2 protein. Subject genetically modified cells that do not produce functional Ptch1 and Ptch2 are insensitive to extracellular Shh exposure, but remain responsive to intracellular Shh (e.g., Shh produced within the cell). The present disclosure provides screening methods to identify agents that modulate the activity of the Hh pathway in a cell.

The present disclosure provides a genetically modified cell (e.g., an in vitro cell), wherein the cell is genetically modified such that the cell does not produce functional Ptch1 and Ptch2 polypeptides. In some cases, the cell is genetically modified such that the cell does not produce Ptch1 and Ptch2 polypeptides. In some cases, the cell is genetically modified with a nucleic acid comprising a nucleotide sequence encoding an Shh protein. In some cases, the Shh protein is a truncated soluble form of Sonic Hedgehog (ShhN). In some cases, the cell is a fibroblast. In some cases, the cell is a mammalian cell. In some cases, the cell is a stem cell (e.g., an embryonic stem cell; an induced pluripotent stem cell; etc.). In some cases, the cell is genetically modified such that the cell is homozygous for a deletion of all or a portion of an endogenous gene encoding Ptch1, and wherein the cell is genetically modified such that the cell is homozygous for a deletion of all or a portion of an endogenous gene encoding Ptch2.

The present disclosure provides a screening method to assess whether a test agent modulates the Hedgehog (Hh) pathway in a cell, the method comprising: a) contacting a genetically modified cell in vitro with a test agent, wherein the genetically modified cell is genetically modified such that it does not produce functional Patched 1 (Ptch1) and Patched 2 (Ptch2), and is genetically modified with an exogenous nucleic acid comprising a nucleotide sequence encoding a Sonic Hedgehog (Shh) polypeptide such that the cell produces the Shh polypeptide intracellularly; and b) determining the effect of the test agent on the Hedgehog (Hh) pathway. In some cases, the cell is a mammalian cell. In some cases, the cell is a fibroblast. In some cases, the encoded Shh is a soluble truncated form of Sonic Hedgehog (ShhN). In some cases, the genetically modified cell is genetically modified with a nucleic acid comprises a nucleotide sequence encoding a reporter polypeptide under the control of a Patch-1 promoter, a Patch-2 promoter, or a Gli-1 promoter, and wherein said determining comprises detecting a level of the reporter polypeptide. In some cases, the reporter protein is an enzyme that catalyzes conversion of a substrate to a detectable reaction product. In some cases, the enzyme is luciferase, β-galactosidase, β-glucuronidase, β-lactamase, alkaline phosphatase, peroxidase, or chloramphenicol acetyltransferase. In some cases, the reporter protein is a fluorescent protein.

### BRIEF DESCRIPTION OF THE DRAWINGS

FIG. 1A-1E provide amino acid sequences of Homo sapiens Patched 1 (Ptch1, isoform L (SEQ ID NO:1); isoform L′ (SEQ ID NO:2); isoform M (SEQ ID NO:3) and isoform S (SEQ ID NO:4)) and the amino acid sequence of Mus musculus Patched 1 (SEQ ID NO:5).

FIG. 2A-2C provide amino acid sequences of Homo sapiens Patched 2 (Ptch2, isoform 1 (SEQ ID NO:6) and isoform 2 (SEQ ID NO:7)) and the amino acid sequence of Mus musculus Patched 2 (SEQ ID NO:8).

FIG. 3A-3B provide amino acid sequences of Homo sapiens and Mus musculus Sonic hedgehog (Shh, Hs (SEQ ID NO:9), Mm (SEQ ID NO:10).

FIG. 4 depicts the net migration of cells transfected with the indicated constructs from six experiments±standard error of the mean to 2 μM purmorphamine which is a Smo agonist used in chemotaxis experiments.

FIG. 5 depicts relative Hh pathway activity in relative luciferase units of Ptch1^{-/-};Ptch2^{-/-} mouse fibroblast cell lines transfected with a Gli-luciferase reporter construct in combination with Ptch1ΔL2, FL Shh, ShhN or Ptch1ΔL2 and ShhN together.

FIG. 6 depicts relative Hh pathway activity in relative luciferase units of reporter mouse fibroblasts co-cultured with empty vector or ShhN transfected mouse fibroblasts.

### DEFINITIONS

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein refers to a nucleic acid or protein that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in and/or produced by a given bacterium, organism, or cell in nature.

“Recombinant,” as used herein, means that a particular nucleic acid or protein is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” nucleic acid or “recombinant” protein refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

By “construct” or “vector” is meant a recombinant nucleic acid, generally recombinant DNA, which has been generated for the purpose of the expression and/or propagation of a nucleotide sequence(s) of interest, or is to be used in the construction of other recombinant nucleotide sequences.

The term “genetically modified” refers to a permanent or transient genetic change in a cell. This may involve deletion of all or a portion of an endogenously encoded gene sequence. e.g., a genetically modified cell that does not produce Ptch1 and Ptch2 may involve deletion of all or a portion of endogenously produced Ptch1 and Ptch2. A genetically modified cell also refers to a cell that no longer produces certain functional proteins as compared to its naturally-occurring state. There are various methods to genetically modify a cell such that it no longer produces certain functional proteins. The choice of method is generally dependent on the type of cell being modified and the circumstances under which the modification is taking place.

The term “transformation” refers to a permanent or transient genetic change induced in a cell following introduction of a nucleic acid (i.e., DNA and/or RNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel et al, Short Protocols in Molecular Biology, 3rd ed., Wiley & Sons, 1995.

The terms “regulatory region” and “regulatory elements”, used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell. As used herein, a “promoter sequence” or “promoter” is a DNA regulatory region capable of binding/recruiting RNA polymerase (e.g., via a transcription initiation complex) and initiating transcription of a downstream (3′ direction) sequence (e.g., a protein coding (“coding”) or non protein-coding (“non-coding”) sequence. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), and/or it may be a temporally restricted promoter (e.g., the promoter is in the “ON” state or “OFF” state during specific stages of embryonic development or during specific stages of a biological process, e.g., hair follicle cycle in mice).

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non protein coding sequence, e.g., a sequence encoding a Shh protein; and the like) if the promoter affects its transcription and/or expression.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a genetically modified cell” includes a plurality of such genetically modified cells and reference to “the test agent” includes reference to one or more test agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a genetically modified cell, wherein the cell is genetically modified such that it does not produce functional Ptch1 and Ptch2 protein. Subject genetically modified cells that do not produce functional Ptch1 and Ptch2 are insensitive to extracellular Shh exposure, but remain responsive to intracellular Shh (e.g., Shh produced within the cell). The present disclosure provides screening methods to identify agents that modulate the activity of the Hh pathway in a cell.

### Genetically Modified Cells

The present disclosure provides a genetically modified cell, wherein the cell is genetically modified such that it does not produce functional Ptch1 and Ptch2 protein Cells that do not produce functional Ptch1 and Ptch2

The present disclosure provides a genetically modified cell such that the cell does not produce functional Ptch1 and Ptch2 protein. In some cases, the genetically modified cell does not produce a Patched 1 (Ptch1) polypeptide or a Patched 2 (Ptch2) polypeptide.

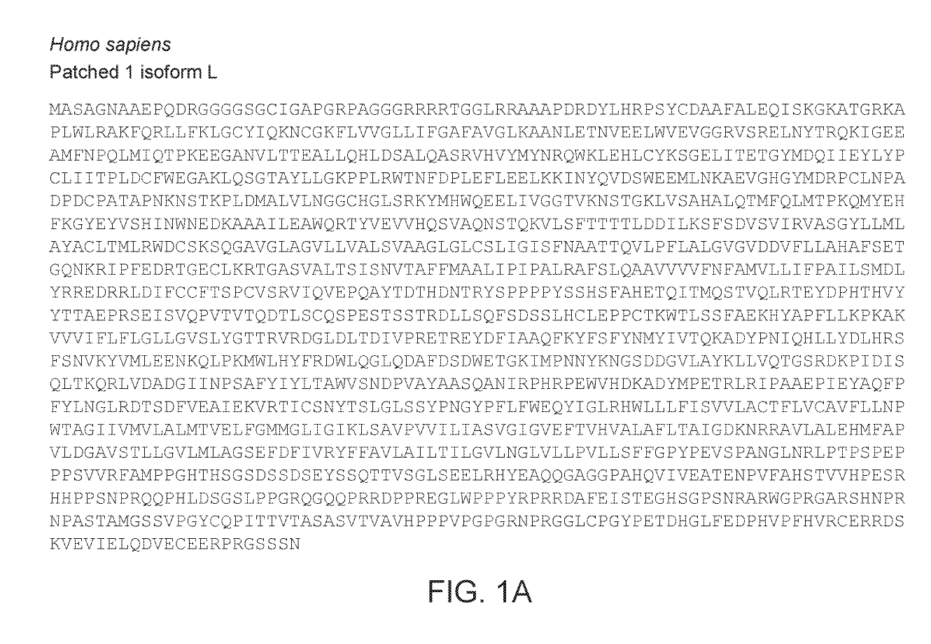

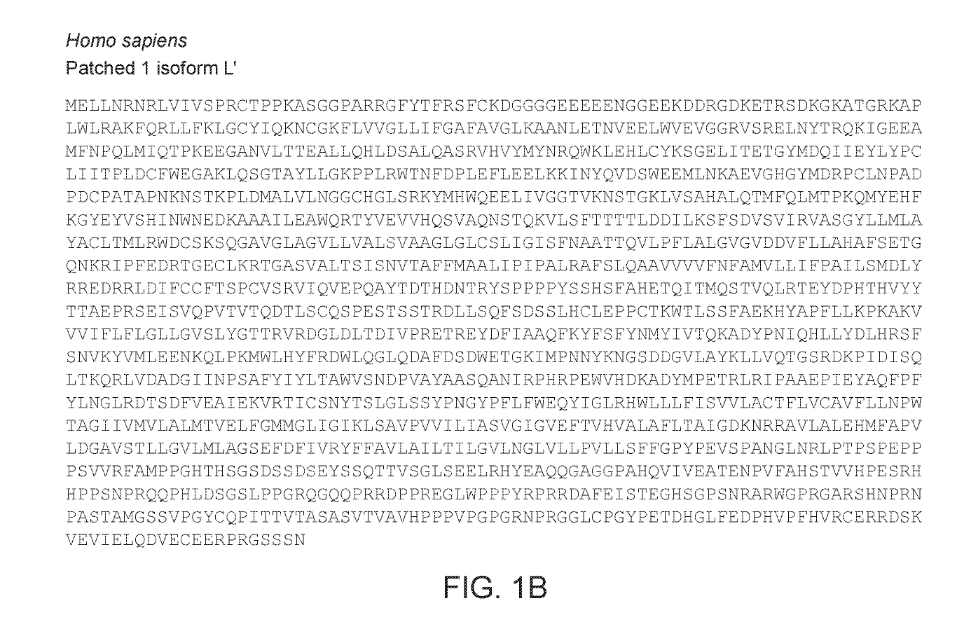

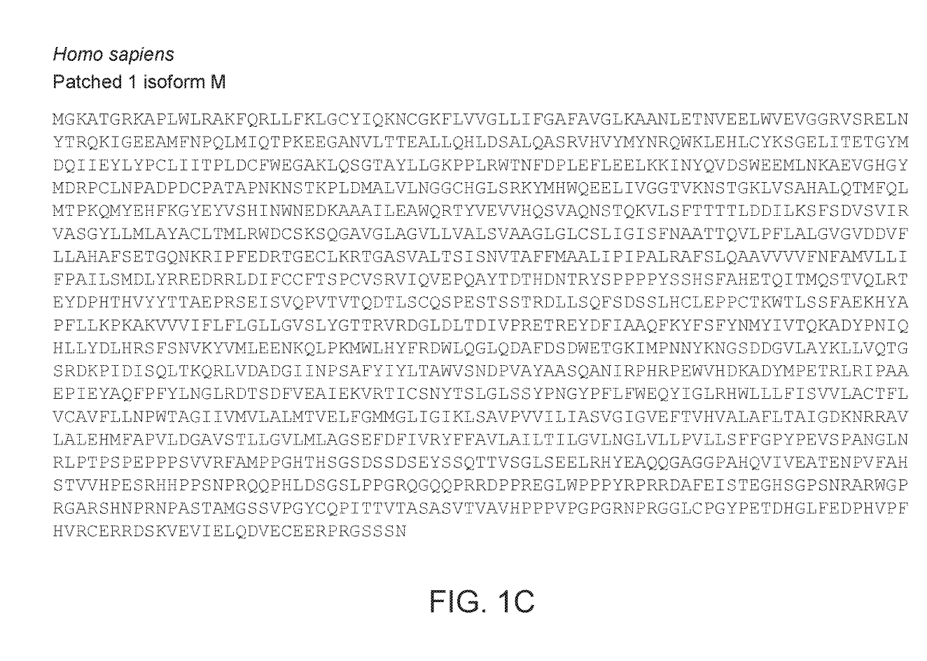

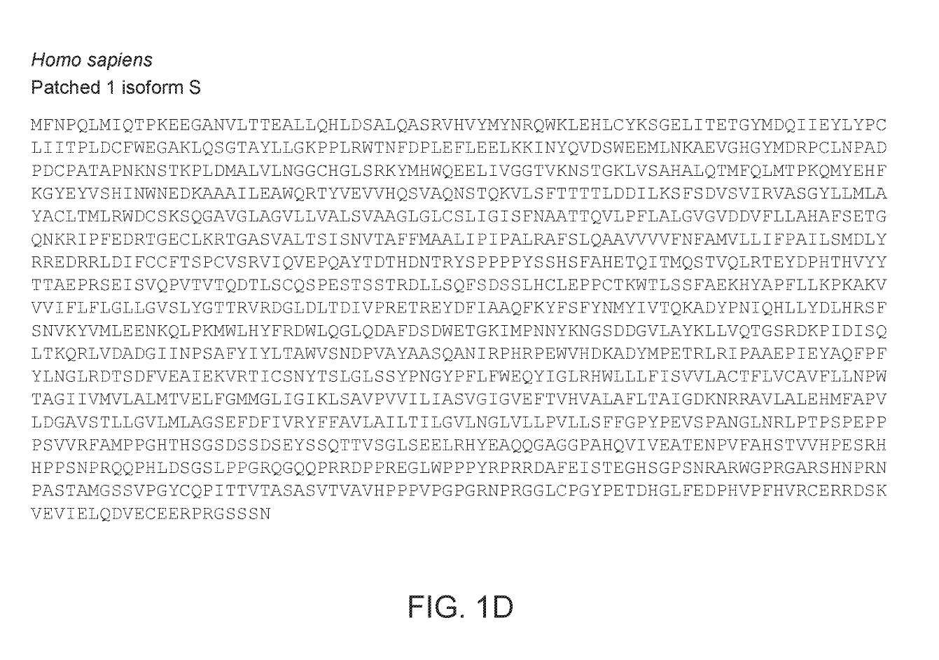

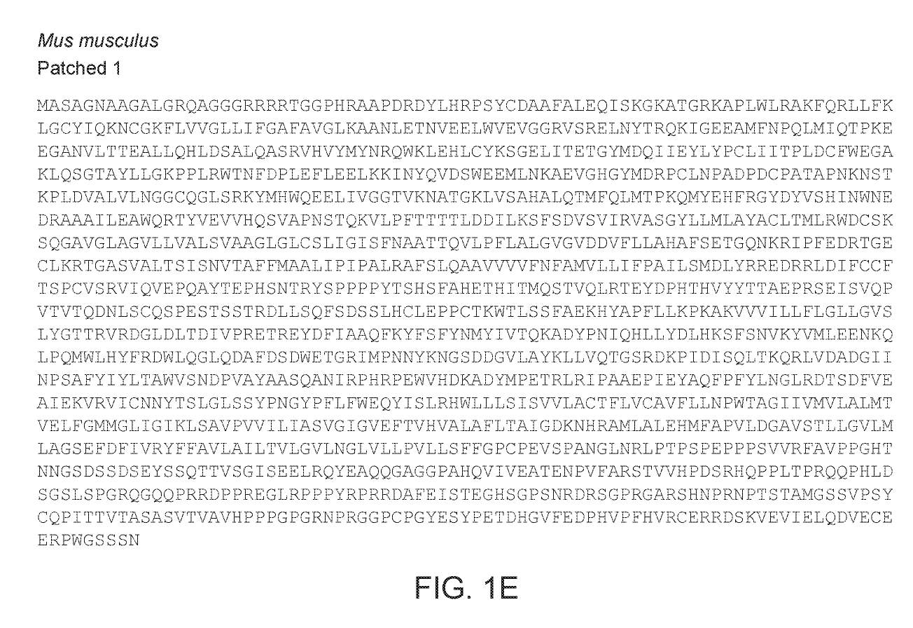

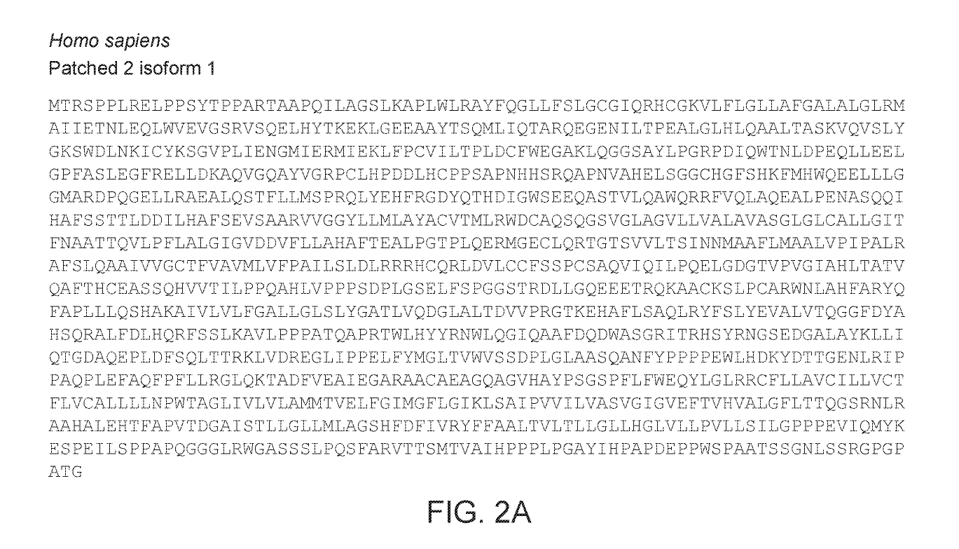

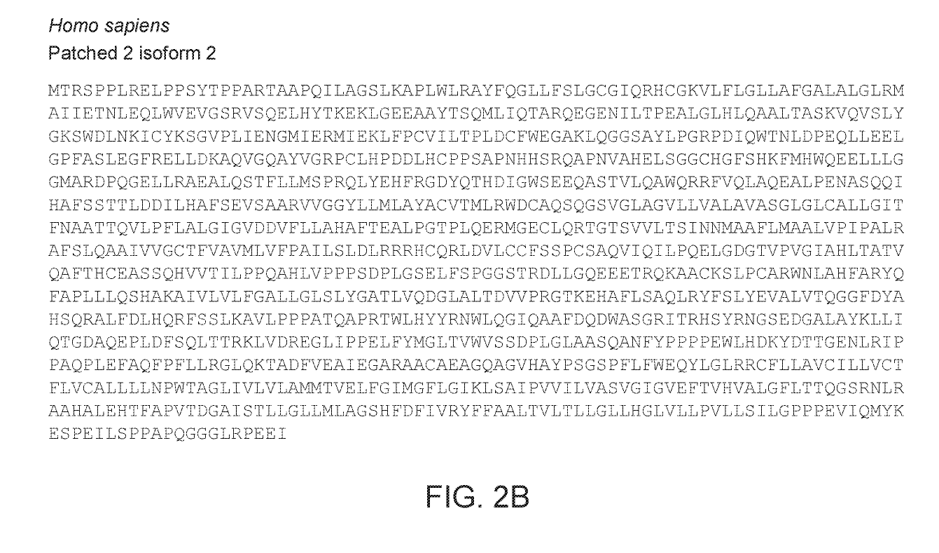

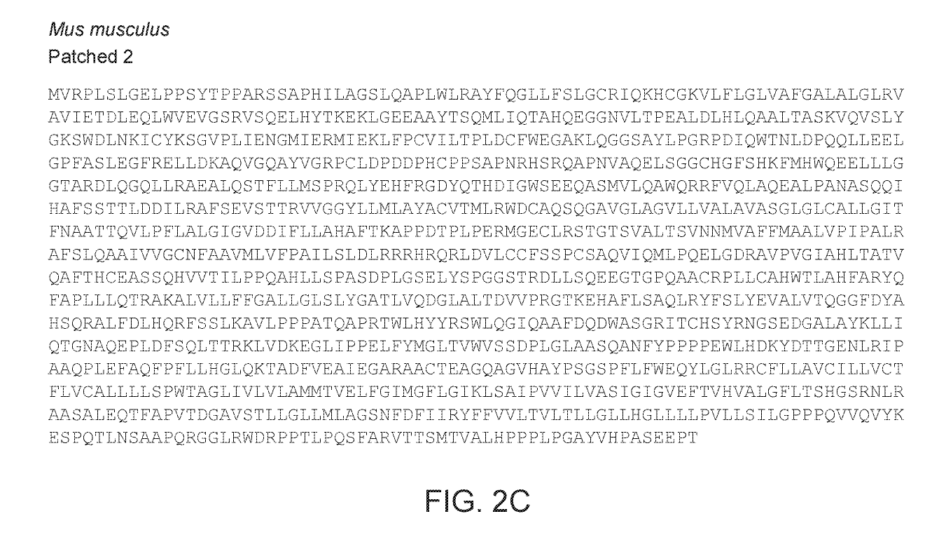

Nucleotide and amino acid sequences of Ptch1 and Ptch2 polypeptides are known in the art. In humans, Ptch1 is alternatively spliced into at least four isoforms and have the amino acid sequences set forth in FIG. 1A-FIG. 1D and SEQ ID NOS:1-4. (Nagao et al., Genomics. 2005, 85:462-471). The nucleotide sequence of the human Ptch1 gene is found under NCBI gene ID 5727. Two human Ptch2 isoforms have the amino acid sequences set forth in FIG. 2A-FIG. 2B and SEQ ID NOS:6-7. (Ranama et al., Biochem J. 2004, 378:325-334). The nucleotide sequence of the human Ptch2 gene is found under NCBI gene ID 8643.

A mouse Ptch1 amino acid sequence is set forth in FIG. 1E and SEQ ID NO:5 (Nagao et al., Genomics. 2005, 85:462-471). Mouse Ptch2 has the amino acid sequence set forth in FIG. 2C and SEQ ID NO:8 (Ranama et al., Biochem J. 2004, 378:325-334). The nucleotide sequence of mouse Ptch1 and Ptch2 genes are found under NCBI gene ID 19206 and 19207, respectively.

As used herein, a “Ptch1” polypeptide encompasses a polypeptide comprising an amino acid sequence having at least 75%, at least 80%, at least 85%, at least 90%, at least 95%, at least 98%, at least 99%, or 100%, amino acid sequence identity to the amino acid sequence set forth in FIG. 1A-1D, FIG. 2A-2B, or FIG. 2C.

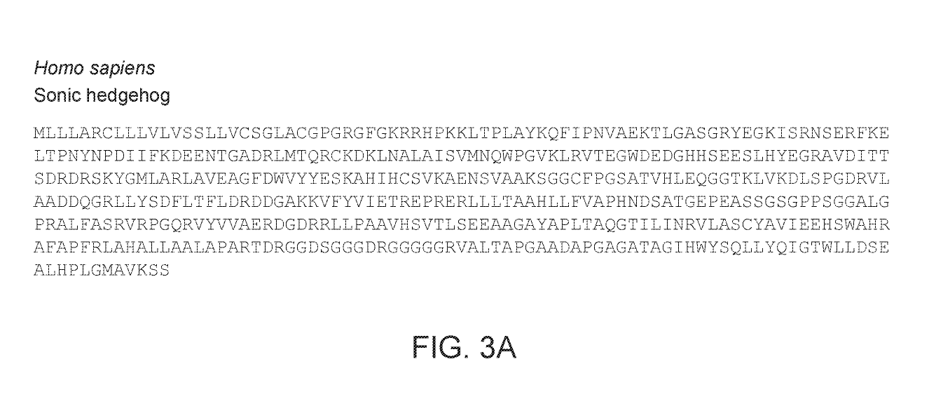

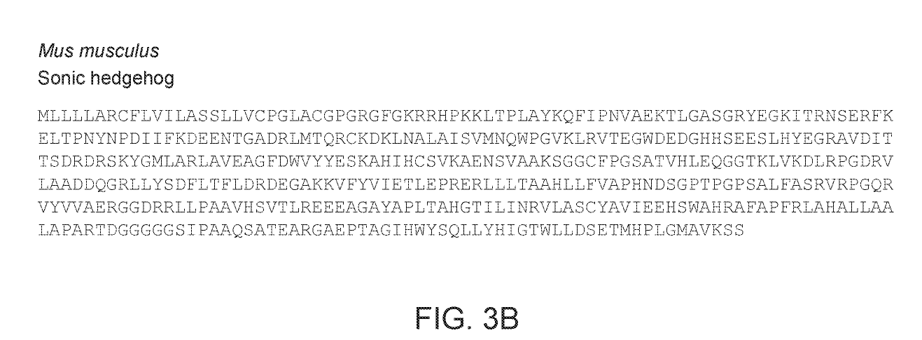

In some cases, a genetically modified cell of the present disclosure is further genetically modified such that it does not produce a functional Sonic Hedgehog (Shh) protein. In some cases the genetically modified cell does not produce a Shh polypeptide. Nucleotide and amino acid sequences of Shh polypeptides are known in the art. Human Shh has the amino acid sequence set forth in FIG. 3A and SEQ ID NO:9 (Belloni et al., Nat Genet. 1996, 14:353-356). Mouse Shh has the amino acid sequence set forth in FIG. 3B and SEQ ID NO:10 (Echelard et al., Cell. 1993, 75:1417-1430).

The nucleotide sequences of the human and mouse Shh genes are found under NCBI gene ID 6469 and 20423, respectively.

In some cases, a genetically modified cell of the present disclosure is genetically modified such that the cell is homozygous for a deletion of all or a portion of an endogenous gene encoding Ptch1, Ptch2 and/or Shh. Methods to delete all or a portion of an endogenous gene are known in the art. In some cases, deletion of all or a portion of an endogenous gene encoding Ptch1, Ptch2 and/or Shh is achieved with the use of transcription activator-like effector nucleases (TALENs) (Cermak et al., Nucleic Acids Res. 2011, 39(12):e82), or use of a CRISPR/Cas9 system. In other cases, a genetically modified cell of the present disclosure is genetically modified such that the cell does not produce Ptch1, Ptch2 and Shh, or such that the cell produces non-functional Ptch1, Ptch2 and Shh, by methods including insertion of a mobile genetic element (e.g., a transposon, etc.); deletion of all or part of the genes, such that the gene products are not made, or is truncated and is non-functional in responding to Shh; mutation of the genes such that the gene products are not made, or is truncated and is non-functional in responding to Shh; deletion or mutation of one or more control elements that control expression of Ptch1, Ptch2 and Shh gene such that the gene products are not made; and the like. Other methods include the use of microRNAs that target mRNA of the target genes for cleavage or repression of productive translation; RNAi; selectively modulating transcription of the genes, and the like.

### Shh Expression

Genetically modified cells of the present disclosure that are genetically modified such that the cell does not produce Ptch1, Ptch2 and/or Shh, are insensitive to exposure to extracellular Shh. This may be due to lack of functional Ptch1 and Ptch2 receptors on the surface of the cell available to bind Shh ligand. In some cases, insensitivity to exposure to extracellular Shh results in no Hedgehog (Hh) pathway activation or response. Methods to determine Hh pathway activation or response are described below.

The present disclosure provides genetically modified cells that are genetically modified such that the cell does not produce Ptch1, Ptch2 and/or Shh, where the genetically modified cells are further genetically modified with a nucleic acid (e.g., an exogenous nucleic acid) comprising a nucleotide sequence encoding a Shh protein. Subject genetically modified cells producing Shh (e.g., exogenous Shh), or variants thereof, within the cells results in activation of the Hh pathway. Thus, a genetically modified cell that is genetically modified such that it does not produce Ptch1 and Ptch2 can respond to intracellular Shh; e.g., intracellular Shh can activate the Hh pathway in the cells.

An Shh protein can be a secreted Hh ligand. Secreted Hh ligands include Shh, Indian Hedgehog (Ihh) and Desert Hedgehog (Dhh), human amino acid sequences of which are known in the art, and set forth in SEQ ID NO:9, SEQ ID NO:11 and SEQ ID NO:12, respectively (Marigo et al., Genomics. 1995, 28:44-51; Kamisago et al., Cytogenet Cell Genet. 1999, 87:117-118). In some cases, a Shh protein of interest may be a variant Shh protein. For example, a variant Shh protein, ShhN, is encoded by a nucleic acid comprising a nucleotide sequence encoding a truncated soluble form of Shh (e.g., residues 24-197 of human Shh (SEQ ID NO:9)) (Bumcrot et al., Mol Cell Biol. 1995, 15:2294-2303; Roelink et al., Cell. 1995, 81:445-455). Variant Shh proteins include but are not limited to ShhN(E90A) and ShhN(H183A) which are mutant variants incapable of binding canonical Shh receptors and co-receptors. Subject genetically modified cells producing ShhN within the cells results in activation of the Hh pathway. Subject genetically modified cells producing ShhN(E90A) or ShhN(H183A) within the cells also results in activation of the Hh pathway.

A nucleic acid encoding Shh or variants thereof, can comprise a nucleotide sequence having 60% or more (e.g., 65% or more, 70% or more, 75% or more, 80% or more, 85% or more, 90% or more, 95% or more, 98% or more, 99% or more, or 100%) nucleotide sequence identity to any of the nucleotide sequences that are found in NCBI gene ID 6469 and 20423. A subject nucleic acid encoding Shh or variants thereof, result in the production of an Shh polypeptide. A Shh polypeptide can comprise amino acid sequence having at least 75%, at least 80%, at least 85%, at least 90%, at least 95%, at least 98%, at least 99%, or 100%, amino acid sequence identity to any of the Shh amino acid sequences set forth in SEQ ID NOS:9-12. In some cases, a nucleic acid encoding a Shh variant, encodes a soluble truncated form of Shh (ShhN), and results in the production of a ShhN polypeptide. A ShhN polypeptide can comprise amino acid sequence having at least 75%, at least 80%, at least 85%, at least 90%, at least 95%, at least 98%, at least 99%, or 100%, amino acid sequence identity to, for example, residues 24-197 of human Shh (SEQ ID NO:9).

In some cases, a nucleic acid encoding Shh or variants thereof is an expression vector, e.g., a recombinant expression vector. In some embodiments, a subject method involves contacting the genetically modified cell with a target nucleic acid or introducing into the genetically modified cell (or a population of cells) (where the cell comprises a target nucleic acid) one or more nucleic acids comprising nucleotide sequences encoding a Shh protein. In some embodiments a cell comprising a target nucleic acid is in vitro. Suitable nucleic acids comprising nucleotide sequences encoding a Shh protein include expression vectors, where an expression vector encoding (comprising a nucleotide sequence encoding) a Shh protein is a “recombinant expression vector.”

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci. 1994, 35:2543-2549; Borras et al., Gene Ther. 1999, 6:515-524; Li and Davidson, Proc Natl Acad Sci USA. 1995, 92:7700-7704; Sakamoto et al., H Gene Ther. 1999, 5:1088-1097; WO 94/12649, WO 93/03769; WO 93/19191; WO 94/28938; WO 95/11984 and WO 95/00655); adeno-associated virus (see, e.g., Ali et al., Hum Gene Ther. 1998, 9:81-86; Flannery et al., Proc Natl Acad Sci USA. 1997, 94:6916-6921; Bennett et al., Invest Opthalmol Vis Sci. 1997, 38:2857-2863; Jomary et al., Gene Ther. 1997, 4:683-690; Rolling et al., Hum Gene Ther. 1999, 10:641-648; Ali et al., Hum Mol Genet. 1996, 5:591-594; Srivastava in WO 93/09239, Samulski et al., J. Vir. 1989, 63:3822-3828; Mendelson et al., Virol. 1988, 166:154-165; and Flotte et al., Proc Natl Acad Sci USA. 1993, 90:10613-10617); SV40; herpes simplex virus; human immunodeficiency virus (see, e.g., Miyoshi et al., Proc Natl Acad Sci USA. 1997, 94:10319-10323; Takahashi et al., J Virol. 1999, 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. Methods in Enzymology. 1987, 153:516-544).

In some embodiments, a nucleotide sequence (e.g., encoding a Shh protein) is operably linked to a regulatory element, e.g., a transcriptional regulatory element, such as a promoter. The transcriptional regulatory element may be functional (operable) in a cell of interest (e.g., a eukaryotic cell, e.g., a mammalian cell; or a prokaryotic cell, e.g., a bacterial or archaeal cell). In some embodiments, a nucleotide sequence (e.g., encoding a Shh protein) is operably linked to multiple control elements that allow expression of the nucleotide sequence encoding a Shh protein.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the subject Shh protein, thus resulting in a chimeric polypeptide.

In some embodiments, a nucleotide sequence encoding a Shh protein is operably linked to an inducible promoter. In other embodiments, a nucleotide sequence encoding a Shh protein is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology. 2002, 20:497-500), an enhanced U6 promoter (e.g., Xia et al., Nucleic Acids Res. 2003, 31(17)), a human H1 promoter (H1), and the like.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germ line, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a Shh protein in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al., Cell. 1987, 51:7-19; and Llewellyn et al., Nat. Med. 2010, 16:1161-1166); a serotonin receptor promoter (see, e.g., GenBank S62283); a tyrosine hydroxylase promoter (TH) (see, e.g., Oh et al., Gene Ther. 2009, 16:437; Sasaoka et al., Mol. Brain Res. 1992, 16:274; Boundy et al., J. Neurosci. 1998, 18:9989; and Kaneda et al., Neuron. 1991, 6:583-594); a GnRH promoter (see, e.g., Radovick et al., Proc. Natl. Acad. Sci. USA. 1991, 88:3402-3406); an L7 promoter (see, e.g., Oberdick et al., Science. 1990, 248:223-226); a DNMT promoter (see, e.g., Bartge et al., Proc. Natl. Acad. Sci. USA. 1988, 85:3648-3652); an enkephalin promoter (see, e.g., Comb et al., EMBO J. 1988, 17:3793-3805); a myelin basic protein (MBP) promoter; a Ca2+-calmodulin-dependent protein kinase II-alpha (CamKIIα) promoter (see, e.g., Mayford et al., Proc. Natl. Acad. Sci. USA. 1996, 93:13250; and Casanova et al., Genesis. 2001, 31:37); a CMV enhancer/platelet-derived growth factor-β promoter (see, e.g., Liu et al., Gene Therapy. 2004, 11:52-60); and the like.

Methods of introducing a nucleic acid into a host cell are known in the art, and any known method can be used to introduce a nucleic acid (e.g., an expression construct) into a cell. Suitable methods include e.g., viral or bacteriophage infection, transfection, conjugation, protoplast fusion, lipofection, nucleofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Kitsune et al., Adv Drug Deliv Rev. 2013, 65:1731-1747), and the like.

Vectors may be provided directly to the subject cells. In other words, the cells are contacted with vectors comprising the nucleic acid encoding a Shh protein such that the vectors are taken up by the cells. Methods for contacting cells with nucleic acid vectors that are plasmids, including electroporation, calcium chloride transfection, microinjection, and lipofection are well known in the art. For viral vector delivery, the cells are contacted with viral particles comprising the nucleic acid encoding a Shh protein. Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also introduced by direct micro-injection.

To generate a genetically modified cell, a construct comprising a nucleotide sequence encoding a Shh protein is introduced stably or transiently into a cell, using established techniques, including, but not limited to, electroporation, calcium phosphate precipitation, DEAE-dextran mediated transfection, liposome-mediated transfection, heat shock in the presence of lithium acetate, and the like. For stable transformation, a nucleic acid will generally further include a selectable marker, e.g., any of several well-known selectable markers such as neomycin resistance, ampicillin resistance, tetracycline resistance, chloramphenicol resistance, kanamycin resistance, and the like.

An Shh protein may be provided to cells as a polypeptide (e.g., introduced into cells as a protein). Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

Additionally or alternatively, the Shh protein may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO:13). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al., Curr Protein Pept Sci. 2003, 4:87-89 and 446; and Wender et al., Proc. Natl. Acad. Sci. U.S.A. 2000, 97:13003-13008; published U.S. Patent applications 20030220334; 20030083256; 20030032593; and 20030022831, herein specifically incorporated by reference for the teachings of translocation peptides and peptoids).

The Shh proteins may be prepared by in vitro synthesis, using conventional methods as known in the art. Various commercial synthetic apparatuses are available, for example, automated synthesizers by Applied Biosystems, Inc., Beckman, etc. The particular sequence and the manner of preparation will be determined by convenience, economics, purity required, and the like.

### Host Cells

Suitable cells for use in generating a genetically modified host cell of the present disclosure include mammalian cells, including primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. Suitable mammalian cell lines include fibroblast cell lines, including but not limited to, BJ cells (ATCC No. CRL-2522), MRC-5 cells (ATCC No. CCL-171), CCD-1112Sk cells (ATCC No. CRL-2429), WS1 cells (ATCC No. CRL-1502), HFL1 cells (ATCC No. CCL-153), and the like.

In some cases, the cell is a stem cell. In some cases, the cell is an induced pluripotent stem cell.

In some embodiments, embryonic stem (ES) cells are used. ES cells are derived from the epiblast of advanced blastocysts. The epiblast cells contribute to all cell types of the developing embryo, rather than the extra-embryonic tissues. Individual ES cells share this totipotency but may be maintained and propagated in an undifferentiated state by culturing them in recombinant leukaemia inhibitory factor (rLIF), or on a monolayer of embryonic fibroblasts which may act as a potent source of this or related cytokines. For ES cells, an ES cell line may be employed, or ES cells may be obtained freshly from a host, e.g. mouse (mESCs), rat, guinea pig, etc.

In some embodiments, cells of mesodermal origin (e.g., fibroblasts) derived from mESCs are used. Methods for deriving cells of mesodermal origin from ES cells are known in the art (see, e.g., Inoue-Yokoo et al., Stem Cell Rev. 2013, 9:422-434).

In other embodiments, embryoid bodies are used. Embryoid bodies are formed from ES cells which have been removed from the inhibitory effects of LIF. The cells proliferate to form clusters of viable cells, each of which represent an embryoid body and can comprise differentiated or partially differentiated cells of a variety of cell types. Neuralized embryoid bodies can be obtained from ES cells that have aggregated in defined medium containing retinoic acid.

The Hh signaling pathway has been shown play a role in tumor initiation as well as progression of tumors to more advanced stages. Aberrant Hh signaling has been found in more than 30% of human cancers, including basal cell carcinoma (BCC), medulloblastoma (MB), melanoma, cancers of the breast, prostate, lung, pancreas, cervix and ovaries. In some embodiments, cancer cell lines (e.g., human basal cell carcinoma cell lines) are used, for example, basal cell carcinoma TE 354.T cells (ATCC No. CRL-7762).

### Screening Methods

The present disclosure provides screening methods for identifying agents that modulate the activity of the Hedgehog (Hh) pathway in a cell.

In some embodiments, the methods are in vitro cell-based screening methods for identifying agents that modulate the activity of the Hh pathway in a cell. In some embodiments, a subject screening method comprises expressing exogenous Shh in a genetically modified cell that does not produce Ptch1 and Ptch2; contacting the genetically modified cell with a test agent; and determining the effect, if any, of the test agent on the activity of the Hh pathway. Methods for genetically modifying a cell such that it not produce Ptch1 and Ptch2, and methods for expressing exogenous Shh in a genetically modified cell have been described above. A reduction in Hh pathway activity, compared to the level of Hh pathway activation in the absence of the test agent, indicates that the test agent reduces the activity of the Hh pathway.

One class of test agent that is of interest is an agent that reduces Hh pathway activity by at least about 10%, at least about 15%, at least about 20%, at least about 25%, at least about 30%, at least about 40%, at least about 50%, at least about 60%, at least about 70%, at least about 80%, or at least about 90%, or more, compared to the level of Hh pathway activation in the absence of the test agent.

In some cases, the present disclosure provides candidate agents that can be further developed into therapeutic agents. In cases where a test agent functions to inhibit the Hh pathway, the test agent can be developed into a therapeutic agent for the treatment of conditions that are the result of increased Hh pathway activity. Such conditions include basal cell carcinoma, medulloblastoma, rhabdomyosarcoma, squamous cell carcinoma, glioma, pericytoma, pancreatic cancer, prostate cancer, breast cancer, colorectal cancer, lung cancer, liver cancer, stomach cancer, and others. As such, a test agent identified using a screening method of the present disclosure, where the test agent reduces Hh pathway activity, is a candidate therapeutic agent for the treatment of basal cell carcinoma, medulloblastoma, rhabdomyosarcoma, squamous cell carcinoma, glioma, pericytoma, pancreatic cancer, prostate cancer, breast cancer, colorectal cancer, lung cancer, liver cancer, stomach cancer, and other conditions.

In some embodiments, an increase in Hh pathway activity, compared to the level of Hh pathway activation in the absence of the test agent, indicates that the test agent increases the activity of the Hh pathway.

Another class of test agent that is of interest is an agent that increases Hh pathway activity by at least about 10%, at least about 15%, at least about 20%, at least about 25%, at least about 30%, at least about 40%, at least about 50%, at least about 60%, at least about 70%, at least about 80%, at least about 90%, at least 2-fold, at least 5-fold, at least 10-fold, or more, compared to the level of Hh pathway activation in the absence of the test agent.

In some cases, the present disclosure provides test agents that can be further developed into therapeutic agents. In cases where a test agent functions to activate the Hh pathway, the test agent can be developed into a therapeutic agent for the treatment of conditions that are the result of insufficient Hh pathway activity. Such conditions include bone disorders, brain disorders, hair loss (e.g., androgenetic alopecia), and others. As such, a test agent identified using a method of the present disclosure, where the test agent activates Hh pathway activity, is a candidate therapeutic agent for the treatment of bone disorders, brain disorders, hair loss (e.g., androgenetic alopecia), and other conditions.

The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Screening may be directed to known pharmacologically active compounds and chemical analogs thereof, or to new agents with unknown properties such as those created through rational drug design.

Candidate agents may be small organic or inorganic compounds having a molecular weight of more than 50 and less than about 10,000 daltons, e.g., from about 50 daltons to about 100 daltons, from about 100 daltons to about 500 daltons, from about 500 daltons to about 1000 daltons, from about 1000 daltons to about 5000 daltons, or from about 5000 daltons to about 10,000 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Screening methods of the present disclosure include controls, where suitable controls include a sample (e.g., a sample comprising the test cell) in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

Agents that have an effect in a screening method of the present disclosure may be further tested for cytotoxicity, bioavailability, and the like, using well known assays. Agents that have an effect in an assay method of the invention may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity and/or ability to cross the blood-brain barrier.

A variety of other reagents may be included in the screening assay. These include reagents like salts, neutral proteins, e.g. albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hour will be sufficient.

A candidate agent is assessed for any cytotoxic activity it may exhibit toward the cell used in the assay, using well-known assays, such as trypan blue dye exclusion, an MTT ([3-(4,5-dimethylthiazol-2-yl)-2,5-diphenyl-2 H-tetrazolium bromide]) assay, and the like. Agents that do not exhibit significant cytotoxic activity are considered candidate agents.

A subject screening method comprises expressing a soluble truncated form of Shh (ShhN) in a genetically modified cell that does not produce Ptch1 and Ptch2; contacting the genetically modified cell with a test agent; and determining the effect, if any, of the test agent on the activity of the Hh pathway. A subject screening method can comprise expressing a soluble mutant truncated form of Shh that is incapable of binding canonical Shh receptors and co-receptors (e.g., ShhN(E90A), ShhN(H183A)).

Another subject screening method comprises expressing a soluble truncated form of Shh (ShhN) in a genetically modified cell that does not produce functional Ptch1 and Ptch2; contacting the genetically modified cell with a test agent; and determining the effect, if any, of the test agent on the activity of the Hh pathway.

In some embodiments, the genetically modified cell of a subject screening method is insensitive to extracellular exposure of Shh. Methods to assess whether the genetically modified cell responds to extracellular exposure of Shh can include, e.g., providing Shh in the culture media, co-culturing cells with cells that secrete Shh, and the like. In some cases, insensitivity to exposure of extracellular Shh results in little to no Hedgehog (Hh) pathway activation or response over a control sample. Methods to determine Hh pathway activation or response are described below.

Any candidate agent identified can be further evaluated, for example, in a secondary screen to determine efficacy in other cell types, to determine cell type specific effects, and the like.

### Reporter Constructs

In some cases, a subject screening method comprises expressing exogenous ShhN in a genetically modified cell that does not produce Ptch1 and Ptch2, further expressing a reporter construct that indicates Hh pathway response, contacting the genetically modified cell with a test agent, and determining the effect of the test agent on Hh pathway response. Detection of the response of the Hh pathway is facilitated by the detection of an Hh pathway reporter construct. In general, reporter constructs of the present disclosure minimally comprise a regulatory element of an Hh pathway gene operably linked to a nucleic acid sequence encoding a detectable reporter protein.

The reporter construct can be introduced into the genetically modified cell according to any of the methods known in the art. The construct can be maintained as an episomal element or integrated into the chromosome of the genetically modified cell. Where the reporter construct is present as a chromosomally integrated element, the reporter construct can utilize a native Hh pathway gene promoter, e.g., the reporter construct can be generated by homologous recombination of a sequence encoding a reporter polypeptide into an Hh pathway gene to provide expression of the reporter polypeptide from the endogenous Hh pathway gene promoter.

Hh pathway genes of interest include genes known in the art to genetically operate downstream of Ptch1 and Ptch2 in response to Shh. Regulatory elements (e.g., promoter, enhancer, response element, etc.) of interest include those that regulate genes that function genetically downstream of Ptch1 and Ptch2. For example, activation of the Hh pathway results in the activation of downstream Gli transcription factors. In some embodiments, Hh pathway response in a genetically modified cell is determined by measuring the response of a Gli transcription factor (e.g., Gli1). In such an embodiment, a recombinant construct that minimally comprises a Gli transcription factor response element (e.g., a Gli transcription factor binding site) operably linked to a nucleic sequence encoding luciferase (e.g., firefly luciferase) is introduced into a genetically modified cell that is genetically modified to not produce Ptch1 and Ptch2. In another embodiment, a recombinant construct that minimally comprises a Gli transcription factor binding site operably linked to a nucleic sequence encoding luciferase (e.g., firefly luciferase) is introduced into a genetically modified cell that is genetically modified to not produce Ptch1 and Ptch2.Upon expression of ShhN within these cells (i.e. activation of the Hh pathway), luciferase reporter activity is modulated and can be quantitatively measured using methods known in the art. In one embodiment, expressing ShhN in a genetically modified cell that does not produce Ptch1 and Ptch2 that additionally expresses a Gli-luciferase reporter construct results in the upregulation of luciferase activity.

In other embodiments, Hh pathway genes of interest include any genes known in the art to be differentially regulated (e.g., upregulated) in response to Shh. For example, Ptch1 and Ptch2 expression is known in the art to be upregulated in response to Shh. As such, additional regulatory elements (e.g., promoter, enhancer, response element, etc.) of interest include those that regulate Ptch1 and Ptch2. For example, in response to Shh, expression of Ptch1 and Ptch2 is upregulated. In some embodiments, Hh pathway response in a genetically modified cell is determined by measuring the response of the Ptch1 promoter driving expression of a detectable reporter. In other embodiments, Hh pathway response in a genetically modified cell is determined by measuring the response of the Ptch2 promoter driving expression of a detectable reporter. In yet another embodiment, Hh pathway response in a genetically modified cell is determined by measuring the response of the Gli1 promoter driving expression of a detectable reporter.

A response element of an Hh pathway component (e.g., Gli transcription factor binding site, Ptch1 promoter, Ptch2 promoter, Gli1 promoter, etc.) that is differentially regulated in response to Shh can be operably linked to nucleic sequences that encode for proteins that generate a detectable signal. Such proteins include protein enzymes capable of catalyzing conversion of a substrate to a detectable reaction product, either directly or indirectly, which have been used, for example, in cell based screening assays. For example, enzymes such as β-galactosidase, β-glucuronidase (GUS), β-lactamase, alkaline phosphatase, peroxidase (e.g., horse radish peroxidase), chloramphenicol acetyltransferase (CAT) and luciferase. Any of a range of enzymes capable of producing a detectable product either directly or indirectly may be so modified or may occur naturally.

β-galactosidase, which is encoded by the E. coli lacZ gene, is an enzyme which has been developed in the art as a reporter enzyme. β-galactosidase activity may be measured by a range of methods including live-cell flow cytometry and histochemical staining with the chromogenic substrate 5-bromo-4-chloro-3-indolyl β-galactopyranoside (X-Gal). Nolan et al., Proc. Natl. Acad. Sci., USA. 2007, 85:2603-2607; and Lojda, Z., Enzyme Histochemistry: A Laboratory Manual, Springer, Berlin, 1979.

In addition to protein enzymes which catalyze a reaction to produce a detectable product, proteins, protein domains or protein fragments which are themselves detectable (e.g., a fluorescent protein) can be used. Exemplary proteins include green fluorescent proteins, which have characteristic detectable emission spectra, and have been modified to alter their emission spectra, as described in PCT WO 96/23810, the disclosure of which is incorporated herein, and fluorescent protein from an Anthozoa species (see, e.g., Matz et al. Nat. Biotechnol. 1999, 17:969-973); and the like. Fusions of fluorescent proteins with other proteins, and DNA sequences encoding the fusion proteins which are expressed in cells are described in PCT WO 95/07463, the disclosure of which is incorporated herein.

Proteins that generate a detectable signal include, but are not limited to, fluorescent proteins, e.g., a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al., J. Protein Chem. 2001, 20:507-519; “humanized” recombinant GFP (hrGFP) (Stratagene); a fluorescent protein as described in U.S. Pat. No. 6,969,597; any of a variety of fluorescent and colored proteins from Anthozoan species, as described in, e.g., Matz et al. Nature Biotechnol. 1999, 17:969-973; and the like. Suitable fluorescent proteins include, e.g., DsRed. See, e.g., Baird et al., Proc Natl Acad Sci USA. 2000, 97:11984-11989. DsRed polypeptides and variants are also described in, e.g., U.S. Patent Publication No. 2005/0244921; and U.S. Pat. No. 6,969,597.

In some embodiments, Hh pathway response can be measured by the level of activation of the Ptch1 promoter operably linked to a nucleic acid that encodes for any of the proteins that generate a detectable signal described above. For example, the Ptch1 promoter operably linked to lacZ.

Other methods known in the art to measure the response of a genetic pathway may be used. For example, using specific phospho-antibodies that detect the phosphorylation status of a known differentially regulated component (e.g., Smoothened) can indicate activation or inhibition of the Hh pathway. In addition, reverse transcription-polymerase chain reaction (RT-PCR) amplification of known downstream target genes (e.g., targets of Gli transcription factors) can be performed to quantitatively measure response of the Hh pathway. Further, RT-PCR of known differentially regulated components of the Hh pathway (e.g., Ptch1,Ptch2, Gli1, etc.) can be performed to quantitatively measure response of the Hh pathway.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Establishment of mESC Lines with Complex Mutant Genotypes for Ptch1, Ptch2, Smo and Shh

A panel of murine embryonic stem cell (mESC) lines was established using genome editing technology. TALENS (Cermak et al., Nucleic Acids Res. 2011, 39(12):e82) were designed targeting Shh, Ptch1, Ptch2, and Smo. The TALEN constructs were transfected singly or in combination into Ptch1^{+/-} or Ptch1^{-/-} (Goodrich et al., Science. 1997, 277:1109-1113) mESC lines. These Ptch1^{+/-} or Ptch1^{-/-} mESC lines were used because they contain LacZ under the control of the Ptch1 promoter, which serves as a genetically encoded measure of the level of Hh pathway activation. The established lines are also optionally mutated for Disp1 (Etheridge et al., Development. 2010, 137:133-140), to further exclude Hh paralogs as mediators of non-autonomous effects.

TALEN constructs were included in a polycystronic message that also encodes antibiotic resistance genes. Transfected mESCs were subjected to high concentration of antibiotics for four days, in the expectation that transient high levels of resistance would be correlated with high levels of TALEN expression. Antibiotic levels were titrated to the point where a few hundred colonies would appear among>10⁶ transfected mESCs. Recovery of mESC colonies harboring predicted non-functional alleles within each of these loci was efficient, and alleles were mutated at frequencies ranging from 5-90%. Due to the transient nature of the expression of the constructs, the cells did not retain resistance to the antibiotics, which allowed the procedures to be repeated thus establishing many mESC lines with various complex genotypes.

This approach was used to demonstrate TALEN-mediated disruption of the Ptch2 locus within Ptch1^{-/-} mESCs. Upon differentiation of the Ptch1^{-/-};Ptch2^{-/-} mESCs into neuralized embryoid bodies (NEBs), the hedgehog response pathway was activated and could not be further induced by the Smo agonist SAG (Chen et al., Proc Natl Acad Sci USA. 2002, 99:14071-14076). In the absence of Ptch1, Ptch2 can mediate some measure of the Shh response. Given the contribution of both Ptch1 and Ptch2 to the interpretation of the Shh signal, it was reasoned that a Ptch1^{-/-};Ptch2^{-/-} genetic background was essential to assess the aggregate contribution of Ptch1/2 to Shh signaling. In agreement with the roles of Ptch1 (and Ptch2) as the Shh receptor, it was found that Ptch1^{-/-};Ptch2^{-/-} differentiate into a highly ventral neural identity, indicated by robust Nk×2.2, Is11/2 and Olig2 staining. It was also found that these cells do not respond to exogenous ShhN, suggesting that Shh signaling is mediated entirely by these two paralogous receptors and that in their absence, Smo is highly active in differentiated tissue independent of Shh activity. The Ptch1^{-/-};Ptch2^{-/-};Shh^{-/-} assessed here have a LacZ under the control of the Ptch1 promoter (Goodrich et al., Science. 1997, 277:1109-1113). Since the upregulation of Ptch1 is a common response to the activation of the Hh response pathway, induction of LacZ in NEBs was assessed. A strong induction of LacZ was observed after three days into the differentiation protocol, consistent with the ventral identity observed using neural markers. At early stages during the differentiation protocol, the level of LacZ was found to be very low, indicating that despite the absence of Ptch1/2, Smo was not activated. This in turn indicates that activation of Smo requires events that are independent of Ptch1/2, but facilitated by the absence of Ptch1/2.

### Example 2

### Ptch1^{-/-};Ptch2^{-/-} Fibroblasts Activate the Transcriptional Shh Pathway in Response to Transfected ShhN

Ptch1^{-/-};Ptch2^{-/-} fibroblast cell lines were derived from Ptch1^{-/-};Ptch2^{-/-} mESCs. Ptch1^{-/-};Ptch2^{-/-} mouse fibroblasts (MFs) show a low level of Hh pathway activity, and are insensitive to Shh exposure. A Gli-luciferase reporter construct was transfected into Ptch^{-/-};Ptch2^{-/-} MFs alone, or in combination with the constitutive inhibitor Ptch1ΔL2, full-length (FL) Shh, ShhN, Ptch1ΔL2 and ShhN together, ShhN(E90A) or ShhN(H183A). Transfected MFs were then grown to confluency and then cultured overnight in low-serum medium. Cells were lysed and the luciferase activity was measured.

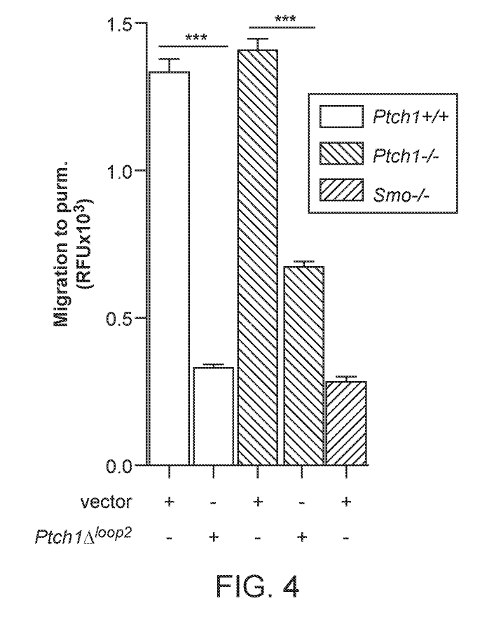

Ptch1ΔL2 is a deletion mutant of Ptch1 that is unable to bind Shh and is a potent inhibitor of the Shh response (Briscoe et al., Mol Cell. 2001, 7:1279-1291). Expression of Ptch1ΔL2 had a strong cell-autonomous inhibitory effect on the Shh response. This is shown in FIG. 4 where the Shh response was measured as net migration from six experiments±standard error of the mean to 2 uM purmorphamine which is a Smo agonist used in chemotaxis experiments. Vector transfected Smo^{-/-} fibroblasts were included as a control.

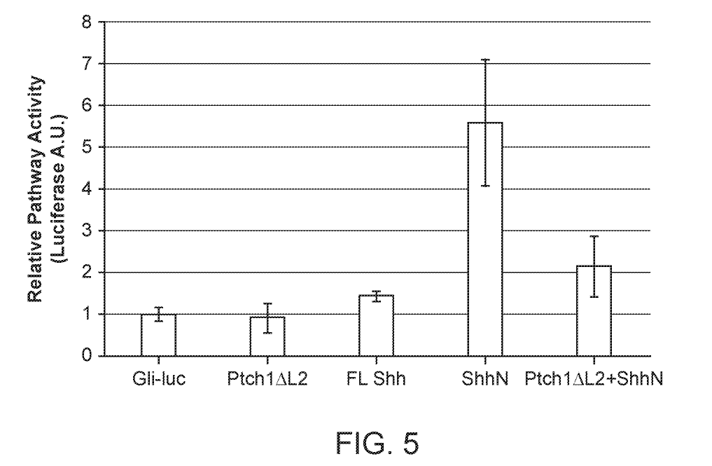

FIG. 5 shows relative Hh pathway activity in relative luciferase units of Ptch1^{-/-}; Ptch2^{-/-} MF cell lines transfected with a Gli-luciferase reporter construct in combination with Ptch1ΔL2, FL Shh, ShhN or Ptch1ΔL2 and ShhN together. Transfection of the soluble truncated ShhN into Ptch1^{-/-};Ptch2^{-/-} MFs induced a strong Hh pathway response. Data shown are mean±standard deviation normalized to the baseline Gli-luciferase activity of Ptch1^{-/-};Ptch2^{-/-} MFs. Transfection of Shh mutants (E90A and H183A) that are unable to bind the canonical Shh receptors or co-receptors were also able to induce Hh pathway response (data not shown). These results demonstrate that Shh can activate Smo cell-autonomously in cells independent of Ptch1 and Ptch2.

### Example 3

### Ptch1^{-/-};Ptch2^{-/-} fibroblasts do not activate the transcriptional Shh pathway in response to extracellular ShhN

Ptch1^{-/-};Ptch2^{-/-} MFs were transfected with a Gli-luciferase reporter construct and co-cultured with Ptch1^{-/-};Ptch2^{-/-} MFs expressing an empty vector (pMES) or ShhN. Co-cultured MFs were grown to confluency and then cultured overnight in low-serum medium. Cells were lysed and the luciferase activity of the reporter cells was measured.

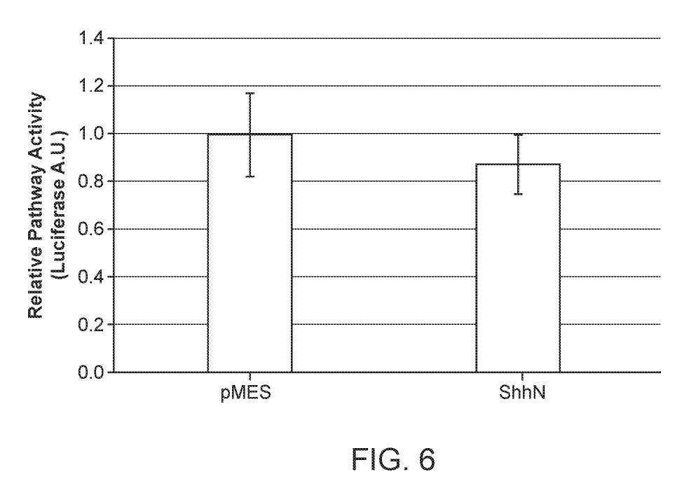

FIG. 6 shows relative Hh pathway activity in relative luciferase units of reporter MFs co-cultured with empty vector or ShhN transfected MFs. Data demonstrates that extracellularly available ShhN did not activate the transcriptional Shh pathway in reporter cells. Data shown are mean±standard deviation normalized to the baseline Gli-luciferase activity of Ptch1^{-/-};Ptch2 ^{-/- MFs.}

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified cell, wherein the cell is genetically modified such that the cell does not produce functional Patched 1 (Ptch1) and Patched 2 (Ptch2) protein.

2. The genetically modified cell of claim 1, wherein the cell is genetically modified such that the cell does not produce Ptch1 and Ptch2 polypeptides.

3. The genetically modified cell of claim 1, wherein the cell is genetically modified with a nucleic acid comprising a nucleotide sequence encoding a Shh protein.

4. The genetically modified cell of claim 3, wherein the Shh protein is a truncated soluble form of Sonic Hedgehog (ShhN).

5. The genetically modified cell of claim 1, wherein the cell is a fibroblast.

6. The genetically modified cell of claim 1, wherein the cell is a mammalian cell.

7. The genetically modified cell of claim 1, wherein the cell is a stem cell.

8. The genetically modified cell of claim 1, wherein the cell is genetically modified such that the cell is homozygous for a deletion of all or a portion of an endogenous gene encoding Ptch1, and wherein the cell is genetically modified such that the cell is homozygous for a deletion of all or a portion of an endogenous gene encoding Ptch2.

9. A screening method to assess whether a test agent modulates the Hedgehog (Hh) pathway in a cell, the method comprising:
a) contacting a genetically modified cell with a test agent, wherein the genetically modified cell is genetically modified such that it does not produce functional Patched 1 (Ptch1) and Patched 2 (Ptch2), and is genetically modified with an exogenous nucleic acid comprising a nucleotide sequence encoding Sonic Hedgehog (Shh); and
b) determining the effect of the test agent on the Hedgehog (Hh) pathway.

10. The method of claim 9, wherein the cell is a mammalian cell.

11. The method of claim 9, wherein the cell is a fibroblast.

12. The method of claim 9, wherein the encoded Shh polypeptide is a soluble truncated form of Sonic Hedgehog (ShhN).

13. The method of claim 9, wherein the genetically modified cell is genetically modified with a nucleic acid comprises a nucleotide sequence encoding a reporter polypeptide under the control of a Patch-1 promoter, a Patch-2 promoter, or a Gli-1 promoter, and wherein said determining comprises detecting a level of the reporter polypeptide.

14. The method of claim 13, wherein said reporter protein is an enzyme that catalyzes conversion of a substrate to a detectable reaction product.

15. The method of claim 14, wherein the enzyme is luciferase, β-galactosidase, β-glucuronidase, β-lactamase, alkaline phosphatase, peroxidase, or chloramphenicol acetyltransferase.

16. The method of claim 13, wherein said reporter protein is a fluorescent protein.


SEQUENCE LISTING (1 sequence)

SEQ ID NO: 1 - protein, 1447 aa, Homo sapiens
MASAGNAAEPQDRGGGGSGCIGAPGRPAGGGRRRRTGGLRRAAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN